(19) United States
(12) Patent Application Publication
(10) Pub. No.: US 2023/0377746 A1
(43) Pub. Date: Nov. 23, 2023

(54) METHOD FOR ESTABLISHING ROBUST PREDICTION MODEL, PREDICTION SYSTEM, AND PROGNOSTIC SYSTEM FOR ALZHEIMER'S DISEASE

(71) Applicant: Kaohsiung Medical University, Kaohsiung (TW)

(72) Inventors: Yuh-Jyh JONG, Kaohsiung City (TW); Yuan-Han YANG, Kaohsiung City (TW); Ming-Chung CHOU, Kaohsiung City (TW); Shin-Mu TSENG, Hsinchu City (TW); Jui-Hung HUNG, Hsinchu City (TW); Hui-Min HSIEH, Kaohsiung City (TW); Shyh-Shin CHIOU, Kaohsiung City (TW)

(21) Appl. No.: 17/855,736

(22) Filed: Jun. 30, 2022

(30) Foreign Application Priority Data
May 18, 2022 (TW) 111118481
Jun. 29, 2022 (TW) 111124343

(51) Int. Cl. G16H 50/20

(52) U.S. Cl. CPC G16H 50/20

(57) ABSTRACT

A method for establishing robust prediction model is adapted for solving the problem that the conventional prediction model cannot generate stable and credible results with missing data. The method of the present invention includes the following steps: obtaining pre-established single-modality standard models respectively based on each type of modalities from samples; extracting modality sets each having the same modality types from the samples to establish corresponding multi-modalities standard models; extracting multiple combinations of the modality sets from the samples having complete modalities to be training data, wherein the multiple combinations of the modality sets can be classified into single-modality, multi-modalities and complete-modalities; inputting said training data into a to-be trained prediction model, and modifying the prediction model by said single-modality standard models and said multi-modalities standard models to obtain a well-trained prediction model.

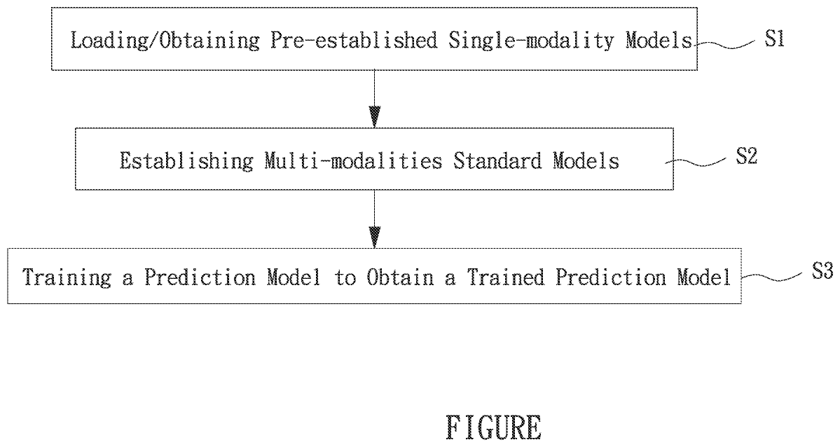

### CROSS REFERENCE TO RELATED APPLICATIONS

The application claims the benefit of Taiwan applications serial No. 111118481, filed May 18, 2022 and serial No. 11124343, filed Jun. 29, 2022, the entire contents of which are incorporated herein by reference.

### BACKGROUND OF THE INVENTION

### 1. Field of the Invention

The present invention relates to a method for establishing a model, and in particular, to a method for establishing a prediction model and a system thereof.

### 2. Description of the Related Art

With the vigorous development of machine learning or artificial intelligence-related theories, algorithms, or models, including but not limited to neural networks, neural-like networks, convolutional neural networks, deep learning, fuzzy theory, randomization, stacking, supervised learning or unsupervised learning, and other content or architectures, machine learning or artificial intelligence has been widely used to establish corresponding prediction models in various fields. In a process of training corresponding models/systems, because corresponding training data needs to be inputted to establish a trained model, the completeness, correctness, and sufficiency of the training data are very important. However, sufficient and adequate training data is not available in all states, and in a state in which the training data is not relatively sufficient, there is still a lack of a method for stably training a corresponding model.

In view of this, it is necessary to improve conventional model training methods.

### SUMMARY OF THE INVENTION

In order to solve the above-mentioned problems, an objective of the present invention is to provide a method for establishing a prediction model, to more completely and accurately train the prediction model and generate a reliable prediction result for input data of different modality combinations.

A secondary objective of the present invention is to provide a method for establishing a prediction model, where more samples/modality data may be generated for establishing standard models, to improve credibility of the corresponding standard models, thereby improving credibility of a subsequent prediction model.

Another objective of the present invention is to provide a prediction system, to provide a stable and credible prediction result.

Still another objective of the present invention is to provide a prognostic system for Alzheimer's disease, to provide a stable and credible prediction result.

The use of the quantifier “a” or “one” in the devices and members described in the entire text of the present invention is only for the convenience of use and provides the usual meaning of the scope of the present invention. In the present invention, it should be interpreted as including one or at least one, and a single concept also includes plural cases unless it clearly explicitly indicates otherwise.

Those of ordinary skill in the art of the present invention may understand that the term “computer” in the present invention refers to various data processing apparatuses having a specific function and implemented by hardware or hardware and software, specifically, is a processor or is provided with a processor, for example, an electronic controller, a server, a cloud platform, a virtual machine, a desktop computer, a notebook computer, a tablet computer, or a smartphone, to process and analyze information and/or generate corresponding control information. In addition, the computer may include a corresponding data receiving or transmitting unit, to receive or transmit required data. In addition, the computer may include a corresponding database/storage unit, to store required data. Especially, unless otherwise specifically excluded or contradicted, the “computer” may be based on “a set of a plurality of computers” in a distributed system architecture and is configured to include or represent a process, a mechanism, and a result of data stream processing between the plurality of computers.

The term “sample” in the present invention refers to any person, event, or object that is observed or of interest and includes information about one type or more types of “modalities”. The term “modality” in the present invention may be any information related to the sample, especially refers to output information/a prediction result generated by inputting any information related to the sample into a pre-established model.

The method for establishing a prediction model in the present invention is performed by a computer. The computer includes at least one processor and at least one storage unit coupled to the processor. The storage unit includes multiple samples, each of the samples includes at least one type of modalities, the samples have N types of modalities in total, and some of the samples each has N types of modalities simultaneously, where N is a positive integer not less than 3. The processor performs the following steps: respectively obtaining C₁^{N} pre-established single-modality standard models according to every single type of the modalities in the samples; obtaining Cₘ^{N} modality combinations having m types of the modalities from the samples having multiple types of modalities to establish Σₙ₌₂^{N-1}Cₙ^{N} corresponding multi-modalities standard models, where m is a combination of positive integers not greater than N−1 and not less than 2; obtaining Σₙ₌₁^{N}Cₙ^{N} modality combinations from the sample having N types of modalities simultaneously to be training data, where the modality combinations in the training data can be classified as single-modality/single-modal/unimodal training data, multi-modalities/multimodal training data and complete-modalities training data; the single-modality training data has C₁^{N} modality combinations in total and the modality combinations have different single types of modalities from each other; the multi-modalities training data has greater than or equal to 2 types and less than or equal to N−1 types of modalities and has Σₙ₌₂^{N-1}Cₙ^{N} modality combinations in total, the modality combinations have different multiple types of modalities from each other, and the complete-modalities training data has a C_{N}^{N} modality combination and has N types of modalities simultaneously; inputting the training data into a to-be-trained prediction model, and modifying the to-be-trained prediction model by using the single-modality standard models and the multi-modalities standard models, to obtain a trained prediction model.

The prediction system of the present invention includes at least one processor and at least one storage unit coupled to the processor. The storage unit has a prediction model, to generate a prediction result for one or more of multiple types of modalities of interest in an input information. The processor receives the input information having the one or more of the multiple types of modalities of interest, and the processor imports the input information to the prediction model to generate the corresponding prediction result.

The prognostic system for Alzheimer's disease of the present invention includes the prediction system, where the multiple types of modalities of interest in the input information are selected from any three types of a clinical factor modality, a brain image modality, an electroencephalographic modality, an environment air pollution modality and a gene modality. Each of the multiple types of modalities of interest is generated by inputting a corresponding characterization information into a corresponding one of the single-modality standard models. The corresponding characterization information refers to at least three of a clinical factor characterization information, a brain image characterization information, an electroencephalographic characterization information, an environment air pollution characterization information and a gene characterization information. The corresponding single-modality standard model refers to at least three of a clinical factor standard model, a brain image standard model, an electroencephalographic model, an environment pollution standard model and a gene standard model.

Based on the foregoing, according to the method for establishing a prediction model, the prediction system, and the prognostic system for Alzheimer's disease of the present invention, a standard model set including single-modality standard models and multi-modalities standard models can be established, and different input data is imported into corresponding standard models to train a prediction model, so that the prediction model can be more completely and more accurately trained. Therefore, a reliable prediction result can be generated for the input data of different modality combinations. In addition, when the multi-modalities standard models are established based on multiple (Cₘ^{N}) modality combinations having multiple types (m) of modalities and obtained from samples having multiple types of modalities (especially the modality combinations can be obtained from data of complete types of modalities or data of a relatively large quantity of types of modalities to form modality data having a target combination, and can be obtained from the modality data having exactly/only the target combination), corresponding multi-modalities standard models can be established and more samples/modality data can be generated for establishing standard models. Therefore, the credibility of the corresponding standard models is improved, and the credibility of a subsequent prediction model is thereby improved

Each of the samples which are used to be the training data, has a corresponding ground truth. When the inputted training data is the single-modality training data or the multi-modalities training data, a ground truth of a corresponding one of the samples is imported. Each of the single types of the modalities or each of the multiple types of the modalities is calculated by using the to-be-trained prediction model to output a prediction result. A corresponding training data is inputted into a corresponding one of the single-modality standard models or a corresponding one of the multi-modalities standard models to generate a corresponding standard result. A corresponding loss function is calculated by using the prediction result, the ground truth and the standard result. When the inputted training data is the complete-modalities training data, each modality in the complete-modalities training data is calculated by the to-be-trained prediction model to output a prediction result, a ground truth of a corresponding one of the samples is imported. A corresponding loss function is calculated by using the prediction result and the ground truth. Therefore, by generating a corresponding loss function according to corresponding different inputted modality combinations (single-modality training data, multi-modalities training data, or complete-modalities training data) and corresponding ground truths and/or standard models (single-modality standard models or multi-modalities standard models), the prediction model can be trained more completely and more accurately. In addition, a reliable prediction result can be generated for the input data of different modality combinations, thereby achieving an effect of improving entire credibility and accuracy of the prediction model.

When the inputted training data is the single-modality training data or the multi-modalities training data, the loss function is defined by a classification loss and a distillation loss. The corresponding classification loss is calculated by using a corresponding prediction result and a corresponding ground truth. The corresponding distillation loss is calculated by using a corresponding prediction result and a corresponding standard result. Therefore, by generating a corresponding classification loss and a corresponding distillation loss according to a corresponding one of the different inputted modality combinations and a corresponding one of the standard models, the prediction model can be trained more completely and more accurately. In addition, a reliable prediction result can be generated for the input data of different modality combinations, thereby achieving the effect of improving the entire credibility and accuracy of the prediction model.

When the inputted training data is the complete-modalities training data, the loss function is defined by a classification loss. The corresponding classification loss is calculated by using a corresponding prediction result and a corresponding ground truth. Therefore, by generating a corresponding classification loss and a corresponding distillation loss according to a corresponding one of the different inputted modality combinations and a corresponding one of the standard models, the prediction model can be trained more completely and more accurately. In addition, a reliable prediction result can be generated for the input data of different modality combinations, thereby achieving the effect of improving the entire credibility and accuracy of the prediction model.

The loss function can be defined as follows:

$${arg min}_{θ} {\sum }_{i}^{c} [ {\sum }_{j}^{{\{ {C}_{hn} \}}_{hn = 1 ∼ N}} {l}_{c l {s}_{j}} ( {\{ {X}_{i}^{k C} \}}_{k = j} , {y}_{i} ; θ ) +$$

$${\sum }_{s}^{{\{ {C}_{h m} \}}_{h m = 1 ∼ m}} {α}_{s} {l}_{{d}_{s}} ( {\{ {X}_{i}^{tC} \}}_{t = s} ; T {e}_{s} ( {ω}_{s} ) ) ]$$

$$where {argmin}_{θ} ( θ )$$

is used for representing that a parameter value θ of the prediction model is calculated by reducing the current loss function to a minimum value; c is used for representing a quantity of samples having complete modalities in training data; i is used for representing a training data sample index value being considered in an epoch; yᵢ is used for representing a ground truth in the training data with a corresponding sample index value; N is used for representing a total quantity of types of modalities; Cₕₙ is used for representing combinations of hn types of modalities selected from N modalities; {Cₕₙ}_{hn=1˜N} is used for representing a set of the combinations of the hn types of modalities from the N types of modalities; j is used for representing a modality combination index value being considered in a classification loss function; l_{cls}_{ⱼ} is used for representing a classification loss function used for calculating a cross entropy loss between an output of a prediction model and ground truth yᵢ, and a subscript j thereof represents a considered modality index value; {Xᵢ^{kC}}ₖ₌ⱼ is used for representing a modality combination j in an i^{th} sample having complete modalities in the training data; θ is used for representing a model parameter of the prediction model; m is used for representing a maximum quantity of multiple types of modalities; Cₕₘ is used for representing combinations of hm types modalities selected from m types of modalities; {Cₕₘ}_{hm=1˜m} is used for representing a set of the combinations of the hm types of modalities selected from the m types of modalities; s is used for representing a modality combination index value being considered in a distillation loss function; l_{d}_{ₛ} is used for representing a distillation loss function, wherein a subscript s thereof represents a considered modality index value; {Xᵢ^{tC}}ₜ₌ₛ is used for representing a modality combination s in the i^{th} sample having the complete modalities in the training data; Teₛ(ωₛ) is used for representing a standard model adapted for calculating the modality combination s; ωₛ is used for representing a model parameter of the standard model adapted for calculating the modality combination s; and αₛ is used for representing a ratio value of each standard model of a corresponding one of modality combinations s in the distillation loss function to an overall loss function of the prediction model, wherein the value is a positive number not less than 0. Therefore, the prediction model can be trained by considering the loss functions of the standard models for different types of modalities, thereby achieving the effect of improving the entire credibility and accuracy of the prediction model.

Based on a teacher-student model training architecture, the single-modality standard models and the multi-modalities standard models are teacher models, and the prediction model is a student model. Therefore, the prediction model may be trained effectively and stably based on teacher-student model training architecture, thereby achieving the effect of improving the entire credibility and accuracy of the prediction model.

In a process of establishing the multi-modalities standard models, the multi-modalities standard models are trained by using a loss function and a gradient descent method, a parameter of a corresponding one of the multi-modalities standard models is modified in each epoch to minimize the loss function, and the trained multi-modalities standard models are established after specified epochs are completed. Therefore, the multi-modalities standard models with high accuracy can be established by minimizing the loss function. Said multi-modalities standard models can be applied to train the prediction model, thereby achieving the effect of improving the entire credibility and accuracy of the prediction model.

The clinical factor characterization information includes at least one of an information of age and gender, a history of related diseases and comorbidity, brain cognitive functions and mental behavior symptoms of a patient. The brain image characterization information includes a magnetic resonance image or a computed tomography image of a brain of a patient. The electroencephalography characterization information includes at least one feature of electroencephalography with a specific frequency and localization. The environment air pollution characterization information is air pollution data of a life place of a patient, where the air pollution data includes at least one of concentration of suspended particulates, concentration of fine suspended particulates, concentration of nitrogen oxides, concentration of nitrogen monoxide, concentration of nitrogen dioxide, concentration of carbon monoxide, concentration of carbon dioxide and concentration of ozone. The gene characterization information is a history of diseases and a single nucleotide polymorphism (SNP) at a specific gene site of a patient. Therefore, the corresponding pre-established single standard models, the multi-modalities models and the prediction model can be established by using at least three of the clinical factor characterization information, the brain image characterization information, the electroencephalography characterization information, the environment air pollution characterization information and the gene characterization information, and the established prediction model can be used for accurate and reliable prognosis of Alzheimer's disease.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

The sole FIGURE is a flowchart of a method according to an exemplary embodiment of the present invention.

### DETAILED DESCRIPTION OF THE INVENTION

The sole FIGURE is an exemplary embodiment of a method for establishing a prediction model according to the present invention. The method is performed by a computer. The computer includes at least one processor and at least one storage unit coupled to the processor. The storage unit includes multiple samples, each of the samples includes at least one type of modalities, the plurality of samples have N types of modalities in total, and some of the samples each has N modalities simultaneously, where N is a positive integer not less than 3. The processor performs the following steps, to establish a stable prediction model.

Step S1: Loading Pre-Established Single-Modality Standard Models.

C₁^{N} pre-established single-modality standard models are respectively obtained/loaded according to every single type of the modalities of the samples. The single-modality standard model refers to that the standard model is related to only a single type modality.

For example, target samples have three (N is equal to 3) types of modalities of interest in total. A corresponding standard model/prediction model may be pre-established for each single modality, and three (C₁^{N}, where N is equal to 3) corresponding single-modality standard models are obtained. More specifically, the three modalities may be respectively a first type modality M1, a second type modality M2 and a third type modality M3, each sample may have three modalities simultaneously (which may be represented, for example, in a vector or matrix manner of [M1, M2, M3]), or may have any two modalities simultaneously (which may be represented, for example, in a vector or matrix manner of [M1, M2, 0], [M1, 0, M3], or [0, M2, M3]), or may have only one modality (which may be represented, for example, in a vector or matrix manner of [M1, 0, 0], [0, M2, 0], or [0, 0, M3]). Namely, having three modalities can be represented, for example, in the vector or matrix manner of [M1, M2, M3]. Similarly, having the first type modality M1 and the second type modality M2 simultaneously can be represented, for example, by [M1, M2, 0], having the first type modality M1 and the third type modality M3 simultaneously can be represented, for example, by [M1, 0, M3], and having the second type modality M2 and the third type modality M3 simultaneously can be represented, for example, by [0, M2, M3]. Having only one of the first, second and third modalities M1, M2 and M3 can be represented, for example, by [M1, 0, 0], [0, M2, 0] and [0, 0, M3], respectively.

For example, for a prognostic model for Alzheimer's disease, the samples may correspond to patients/observed persons. The modality may be a prediction result obtained by inputting characterization information of the patient (sample) related to Alzheimer's disease into a corresponding standard model. The characterization information may be, for example, at least one of a clinical factor, a brain image factor, an environment air pollution factor, or a gene factor, and is not to limit the present invention. The standard model is a pre-established standard model and is configured to generate a corresponding prediction result (a modality) after a corresponding characterization information is inputted.

The standard model may be established by using one or more of machine learning or artificial intelligent-related theories, algorithms, or models and through a specific model training process such as data preparation, model selection, optional application of a specific architecture theory, model/machine training (outputting a result), result evaluation and analysis (comparing the outputted result with a more accurate result or ground truth, and generating an optimization direction by using a corresponding algorithm or logic), parameter/hyper-parameter adjustment (adjusting a parameter according to the optimization direction), performing a plurality of times of training optionally, completing model training, and performing prediction through a completed model thereby. The process of establishing the standard model is merely exemplary and belongs to an ordinary skill in the technical field of the present invention and is not to limit the present invention.

Step S2: Establishing Multi-Modalities Standard Models

Cₘ^{N} modality combinations/data types having m types of the modalities are obtained from the samples having multiple types of modalities, to establish Σₙ₌₂^{N-1}Cₙ^{N} corresponding multi-modalities standard models, where m is a combination of positive integers not greater than N−1 and not less than 2, which may be represented as a mathematical expression of {m|2≤m≤N−1}. The multi-modalities standard model refers to that the standard model is related to the multiple types of the modalities, and the multiple types of the modalities do not include complete modalities. The term “complete modalities” refers to the modality combination having all (N) types of the modalities in the sample.

Continuing with the example in step S1, for example, target samples have three (N is equal to 3) types of the modalities of interest. The three types of the modalities may be respectively a first type modality M1, a second type modality M2, and a third type modality M3, and two (that is, m is equal to 2) types of the modalities may be obtained from a sample having three types of the modalities or a sample having two types of the modalities simultaneously. That is, if a data type of [M1, M2, 0] needs to be obtained to form training data, a non-related modality can be removed from a sample having three types of the modalities [M1, M2, M3] simultaneously (that is, a third type modality M3 is replaced by 0) to form the corresponding data type, and a sample originally having exact modalities corresponding to a data type of [M1, M2, 0] is also applied, so that a corresponding multi-modalities standard model is established for the data type of [M1, M2, 0]. Similarly, non-related modalities can be removed from a sample having three types of the modalities simultaneously in the foregoing manner, and samples having two corresponding types of the modalities are also applied, to form training data, so that multi-modalities standard models are respectively established for data types of [M1, 0, M3] and [0, M2, M3]. In this case, there are three (cₘ^{N}, where N=3 and m=2) data types of [M1, M2, 0], [M1, 0, M3], and [0, M2, M3] in total, and three (Σₙ₌₂^{N-1}Cₙ^{N}, where N=3) corresponding multi-modalities standard models may be respectively established.

In another example, if target samples have four (N is equal to 4) types of modalities of interest in total, the four types of modalities may be respectively a first type modality to a fourth type modality M1 to M4, six (Cₘ^{N}, where N=4 and m=2) combinations having two (that is, m is equal to 2) types of the modalities can be obtained from the sample, and four (Cₘ^{N}, where N=4 and m=3) combinations having three (that is, m=3) types of the modalities can be obtained from the samples. Specifically, in the state for obtaining two types of the modalities, the corresponding modality combinations are [M1, M2, 0, 0], [M1, 0, M3, 0], [M1, 0, 0, M4], [0, M2, M3, 0], [0, M2, 0, M4], and [0, 0, M3, M4]. In the state for obtaining three types of the modalities, the corresponding modality combinations are [M1, M2, M3, 0], [M1, M2, 0, M4], [M1, 0, M3, M4], and [0, M2, M3, M4].

Specifically, during establishment of the multi-modalities standard models, each multi-modalities standard model having two types of modalities (for example, [M1, M2, 0] or [M1, M2, 0, 0]) is related to each single-modality standard model having one of the two modalities (M1 or M2), and each multi-modalities standard model having m+1 types of modalities is related to each multi-modalities standard model having m types of modalities of the m+1 types of modalities, to train a corresponding multi-modalities standard model. In other words, a current multi-modalities standard model having multiple types of modalities is trained by using a standard model having one fewer modality type. For example, target samples have three types of modalities of interest in total. If a multi-modalities model having two types of modalities such as [M1, M2, 0] is trained, standard models (that is, single-modality standard models) having one fewer modality type (that is, one modality) [M1, 0, 0] and [0, M2, 0] are selected for training the multi-modalities standard model. For example, target samples have four types of modalities of interest in total. Firstly, for training a multi-modalities model having two types of modalities such as [M1, M2, 0, 0], standard models (that is, single-modality standard models) having one fewer modality type (that is, one modality) [M1, 0, 0, 0] and [0, M2, 0, 0] are selected for training the multi-modalities standard model with two types of modalities. Subsequently, for training a multi-modalities model having three types of modalities such as [M1, M2, M3, 0], standard models (that is, multi-modalities standard models) having one fewer modality type (that is, two modalities) [M1, M2, 0, 0], [M1, 0, M3, 0], and [0, M2, M3, 0] are selected for training the multi-modalities standard model with three types of modalities.

Optionally, during establishment of the multi-modalities standard models, the multi-modalities standard models are trained by using a loss function and a gradient descent method. Thereby, a parameter of a corresponding model is modified/adjusted in each epoch, to minimize the loss function, and the trained multi-modalities standard models are established after specified epochs are completed/ended. Preferably, the loss function includes a classification loss and a distillation loss. In a calculation of the classification loss, some of the plurality of samples each has N types of modalities and a corresponding ground truth, and the classification loss is calculated by using the ground truth. In a calculation of the distillation loss, the distillation loss is calculated by using a standard result/dark knowledge (for example, a probability) outputted by a corresponding single-modality standard model. More preferably, the multi-modalities standard model generates an output data and an output probability. The classification loss is calculated by using the output data and a corresponding ground truth, and the distillation loss is calculated by using the output probability and a corresponding standard result.

Step S3: Training a Prediction Model

Σₙ₌₁^{N}Cₙ^{N} modality combinations are obtained from the samples having N types of modalities simultaneously as training data, where the modality combinations in the training data can be classified as single-modality training data, multi-modalities training data, and complete-modalities training data. The single-modality training data has C₁^{N} modality combinations and the modality combinations have different single types of modalities from each other. The multi-modalities training data has greater than or equal to 2 types and less than or equal to N−1 types of modalities and has Σₙ₌₂^{N-1}Cₙ^{N} modality combinations in total, and the modality combinations have a plurality of different modalities from each other. The complete-modalities training data has a C_{N}^{N} modality combination and has the N types of modalities simultaneously. The training data is inputted into a to-be-trained prediction model, and the to-be-trained prediction model is modified by using the single-modality standard models and the multi-modalities standard models, to obtain a trained prediction model. Optionally, based on a teacher-student model training architecture, the single-modality standard models and the multi-modalities standard models are teacher models, and the prediction model is a student model. A set of the standard models includes a plurality of different teacher models.

Continuing with the examples in step S1 and step S2, similarly, target samples have three (N is equal to 3) types of modalities of interest in total, and the three types of modalities can be respectively a first type modality to a third type modality M1 to M3. Single-modality training data can be obtained from the samples, which is respectively single types of modalities of [M1, 0, 0], [0, M2, 0], and [0, 0, M3], and has three (C₁^{N}, where N=3) types of modality combinations in total. Multi-modalities training data can be obtained from the samples. That is, modality combinations having two types of the modalities can be obtained, which may be represented as [M1, M2, 0], [M1, 0, M3], and [0, M2, M3], and has three (Σₙ₌₂^{N-1}₂^{N}C, where N=3) corresponding modality combinations in total. Complete-modalities training data can be obtained from the samples, that is, a modality combination having three types of the modalities (that is, N modalities) simultaneously can be obtained, which may be represented as [M1, M2, M3], and has one (C_{N}^{N}) corresponding modality combination.

Similarly, for example, target samples have four (N is equal to 4) types of modalities of interest in total, and the four types of modalities may be respectively a first type modality to a fourth type modality M1 to M4. Single-modality training data can be obtained from the samples, which is respectively single types of modalities [M1, 0, 0, 0], [0, M2, 0, 0], [0, 0, M3, 0], and [0, 0, 0, M4], and has four (C₁^{N}, where N=4) corresponding modality combinations in total. Multi-modalities training data can be obtained from the samples, that is, modality combinations having two types of the modalities and three types of the modalities simultaneously can be obtained, which may be represented as [M1, M2, 0, 0], [M1, 0, M3, 0], [M1, 0, 0, M4], [0, M2, M3, 0], [0, M2, 0, M4], [0, 0, M3, M4], [M1, M2, M3, 0], [M1, M2, 0, M4], [M1, 0, M3, M4], [0, M2, M3, M4], and has 10 (Σₙ₌₂^{N-1}Cₙ^{N}, where N=4) corresponding modality combinations in total. Complete-modalities training data can be obtained from the samples, that is, a modality combination having four types of the modalities (that is, N modalities) simultaneously can be obtained, which may be represented as [M1, M2, M3, M4], and has one (C_{N}^{N}) corresponding modality combination.

Preferably, during establishment/training of the prediction model, the prediction model is trained by using a loss function and a gradient descent method. A parameter of a corresponding model is modified/adjusted in each epoch to minimize the loss function, and the trained prediction model is established after specified epochs are completed/ended.

Specifically, each of the samples, as the training data, has a corresponding ground truth. When inputted training data is the single-modality training data or the multi-modalities training data, each of the different single types of modalities or each of the multiple types of modalities is calculated by using the to-be-trained prediction model to output a prediction result, a ground truth of a corresponding sample is imported, and the training data is inputted into a corresponding single-modality standard model and/or a corresponding multi-modalities standard model to generate a corresponding standard result. Thereby, a corresponding loss function is calculated by using the prediction result, the ground truth and the standard result.

Specifically, each of the samples, as the training data, has a corresponding ground truth. When inputted training data is the complete-modalities training data, each modality in the complete-modalities training data is calculated by using the to-be-trained prediction model to output a prediction result, the ground truth of a corresponding sample is imported. Thereby a corresponding loss function is calculated by using the prediction result and the ground truth.

Preferably, when the inputted training data is the single-modality training data or the multi-modalities training data, the loss function is defined by a classification loss and a distillation loss. The corresponding classification loss is calculated by using a corresponding prediction result and a corresponding ground truth. The corresponding distillation loss is calculated by using a corresponding prediction result and a corresponding standard result. Specifically, during calculation of the distillation loss, corresponding training data is inputted into a corresponding one of the standard models (that is, a corresponding one of the single-modality standard models or a corresponding one of the multi-modalities standard models), to obtain a corresponding standard result/dark knowledge, and the distillation loss is calculated by using a corresponding prediction result and the standard result. More preferably, the corresponding prediction result includes prediction data and a prediction probability. The classification loss is calculated by using the prediction data and a corresponding ground truth, and the distillation loss is calculated by using the prediction probability and a corresponding standard result.

Preferably, when the inputted training data is the complete-modalities training data, the loss function is defined by a classification loss. The corresponding classification loss is calculated by using a corresponding prediction result and a corresponding ground truth. More preferably, the corresponding prediction result includes prediction data. The classification loss is calculated by using the prediction data and a corresponding ground truth.

For example, target samples have three types of modalities of interest in total. If inputted training data is single-modality training data (for example, [M1, 0, 0]), a single-modality standard model is imported for calculating the training data (for example, [M1, 0, 0]) to generate a corresponding standard result. Therefore, a corresponding distillation loss is calculated by using the standard result, and a classification loss is calculated by using a corresponding ground truth. If the inputted training data is multi-modalities training data (for example, [M1, M2, 0]), a multi-modalities standard model is imported for calculating the training data (for example, [M1, M2, 0] to generate a corresponding standard result. Therefore, a corresponding distillation loss is calculated by using the standard result, and a classification loss is calculated by using a corresponding ground truth. If the inputted training data is complete-modalities training data (that is, [M1, M2, M3]), there is no corresponding standard model, so that there is no distillation loss, and a classification loss is calculated according to a corresponding ground truth.

Specifically, in an example, the loss function of the prediction model may be defined as follows:

$${arg min}_{θ} {\sum }_{i}^{c} [ {\sum }_{j}^{{\{ {C}_{hn} \}}_{hn = 1 ∼ N}} {l}_{c l {s}_{j}} ( {\{ {X}_{i}^{k C} \}}_{k = j} , {y}_{i} ; θ ) +$$

$${\sum }_{s}^{{\{ {C}_{h m} \}}_{h m = 1 ∼ m}} {α}_{s} {l}_{{d}_{s}} ( {\{ {X}_{i}^{tC} \}}_{t = s} ; T {e}_{s} ( {ω}_{s} ) ) ]$$

$$where {argmin}_{θ} ( θ )$$

is used for representing that a parameter value 9 of the prediction model is calculated by reducing the current loss function to a minimum value; c is used for representing a quantity of samples having complete modalities in training data; i is used for representing a training data sample index value being considered in an epoch; yᵢ is used for representing a ground truth in the training data with a corresponding sample index value; N is used for representing a total quantity of types of modalities; Cₕₙ is used for representing combinations of hn types of modalities selected from N types of modalities; {Cₕₙ}_{hn=1˜N} is used for representing a set of the combinations of the hn types of modalities from the N types of modalities (for example, when N=3, h=2, {C₂}={{12}, {13}, {23}}, where {12} represents [M1, M2, 0] as shown in the above-mentioned contents); j is used for representing a modality combination index value being considered in a classification loss function; l_{cls}_{ⱼ} is used for representing a classification loss function used for calculating a cross entropy loss between an output of a prediction model and ground truth yᵢ, where a subscript j thereof represents a considered modality index value; {Xᵢ^{kC}}ₖ₌ⱼ is used for representing a modality combination j in an i^{th} sample having complete modalities in the training data (for example, when j={1,2}, {Xᵢ^{kC}}_{k={1,2}}={Xᵢ^{1C},Xᵢ^{2C}}); θ is used for representing a model parameter of the prediction model; m is used for representing a maximum quantity of multiple types of modalities; Cₕₘ is used for representing combinations of hm types of modalities selected from m types of modalities; {Cₕₘ}_{hm=1˜m} is used for representing a set of the combinations of the hm types of modalities selected from the m types of modalities; s is used for representing a modality combination index value being considered in a distillation loss function; l_{d}_{ₛ} is used for representing a distillation loss function, where a subscript s thereof represents a considered modality index value; {Xᵢ^{tC}}ₜ₌ₛ is used for representing a modality combination s in the i^{th} sample having the complete modalities in the training data (for example, when s={1,2}, {Xᵢ^{tC}}_{t={1,2}}={Xᵢ^{1C},Xᵢ^{2C}}); Teₛ(ωₛ) is used for representing a standard model adapted for calculating/considering the modality combination s; ωₛ is used for representing a model parameter of the standard model adapted for calculating/considering the modality combination s; and αₛ is used for representing a ratio value of the standard model of the modality combination s in the distillation loss function to an overall loss function of the prediction model, where the value is a positive number not less than 0. Particularly, the calculation of the distillation loss function refers to the calculation of a Kullback-Leibler divergence between an output of a prediction model and outputs of standard models Teₛ(ωₛ). The output of the prediction model and the outputs of the standard models are classification probability distribution obtained by adding a temperature/smoothing parameter to a softmax function.

According to the above-mentioned loss function, samples having three (N=3) types of modalities in total are used as an example, the loss function of the prediction model is presented as follows after being expanded:

$${arg min}_{θ} {\sum }_{i}^{c} {l}_{c l {s}_{1}} ( {X}_{i}^{1 C} , {y}_{i} ; θ ) +$$

$${l}_{c l {s}_{2}} ( {X}_{i}^{2 C} , {y}_{i} ; θ ) +$$

$${l}_{{cls}_{3}} ( {X}_{i}^{3 C} , {y}_{i} ; θ ) +$$

$${l}_{{cls}_{12}} ( {X}_{i}^{1 C} , {X}_{i}^{2 C} , {y}_{i} ; θ ) +$$

$${l}_{{cls}_{13}} ( {X}_{i}^{1 C} , {X}_{i}^{3 C} , {y}_{i} ; θ ) +$$

$${l}_{{cls}_{23}} ( {X}_{i}^{2 C} , {X}_{i}^{3 C} , {y}_{i} ; θ ) +$$

$${l}_{{cls}_{123}} ( {X}_{i}^{1 C} , {X}_{i}^{2 C} , {X}_{i}^{3 C} , {y}_{i} ; θ ) +$$

$${α}_{1} {l}_{{d}_{1}} ( {X}_{i}^{1 C} ; T {e}_{1} ( {ω}_{1} ) ) +$$

$${α}_{2} {l}_{{d}_{2}} ( {X}_{i}^{2 C} ; T {e}_{2} ( {ω}_{2} ) ) +$$

$${α}_{3} {l}_{{d}_{3}} ( {X}_{i}^{3 C} ; {Te}_{3} ( {ω}_{3} ) ) +$$

$${α}_{12} {l}_{{d}_{12}} ( {X}_{i}^{1 C} , {X}_{i}^{2 C} ; T {e}_{12} ( {ω}_{12} ) ) +$$

$${α}_{13} {l}_{{d}_{13}} ( {X}_{i}^{1 C} , {X}_{i}^{3 C} ; T {e}_{13} ( {ω}_{13} ) ) +$$

$${α}_{23} {l}_{{d}_{23}} ( {X}_{i}^{2 C} , {X}_{i}^{3 C} ; T {e}_{23} ( {ω}_{23} ) )$$

where Xᵢ^{1C} is used for representing a first type modality in the samples having complete modalities in training data; Xᵢ^{2C} i is used for representing a second type modality in the samples having the complete modalities in the training data; Xᵢ^{3C} is used for representing a third type modality in the samples having the complete modalities in the training data; each one of l_{cls}_{₁}, l_{cls}_{₂}, l_{cls}_{₃}, l_{cls}_{₁₂}, l_{cls}_{₁₃}, l_{cls}_{₂₃} and l_{cls}_{₁₂₃} is used for representing a classification loss function, where subscript thereof represent considered modality index value, and an index value set is [1, 2, 3, 12, 13, 23, 123]; for example, marking index value [12] indicates that only a first type modality and a second type modality are considered, and marking index value [123] indicates that a first type modality, a second type modality and a third type modality are considered; Each one of Te₁(ω₁), Te₂(ω₂), Te₃(ω₃), Te₁₂(ω₁₂), Te₁₃(ω₁₃) and Te₂₃(ω₂₃) is used for representing a standard model, where Te₁(ω₁), Te₂(ω₂) and Te₃(ω₃) are single-modality standard models, Te₁₂(ω₁₂), Te₁₃(ω₁₃) and Te₂₃(ω₂₃) are multi-modalities standard models, ω₁, ω₂ and ω₃ are model parameters of the single-modality standard models, and ω₁₂, ω₁₃ and ω₂₃ are model parameters of the multi-modalities standard models; each one of l_{d}_{₁}, l_{d}_{₂}, l_{d}_{₃}, l_{d}_{₁₂}, l_{d}_{₁₃} and l_{d}_{₂₃} are distillation loss functions, where subscripts thereof represent considered modality index values, and an index value set is [1, 2, 3, 12, 13, 23]; for example, marking index values [12] indicates that only a first type modality and a second type modality are considered; and each one of α₁, α₂, α₃, α₁₂, α₁₃ and α₂₃ is used for representing a ratio value the respective one of the distillation loss functions to an overall loss function of the prediction model, where the value is a positive number not less than 0.

Forming a Prediction System by Using the Trained Prediction Model

Based on the foregoing, the present invention provides a prediction system, including at least one processor and at least one storage unit coupled to the processor. The storage unit has a prediction model, and the prediction model is established according to the above-mentioned method for establishing a prediction model to generate a prediction result for one or more of a plurality of modalities of interest in an input information. The processor receives the input information having the one or more of the plurality of modalities of interest and imports the input information to the prediction model to generate the corresponding prediction result. In other words, after the prediction model is established, information (including same or different information in the prediction model establishment process) having corresponding modalities of interest in any sample can be inputted into the prediction model to generate a corresponding prediction result. Therefore, by applying the prediction system established by the prediction model, in the absence of input data/modalities (there are only partial/incomplete input data/modalities; particularly indicating the state that the complete modalities have N types of modalities, and the input data has a modality/modalities with types less than N), a stable and credible prediction result can be still obtained.

Preferably, the prediction result is a two-dimensional vector, a first dimension represents non-deterioration, and a second dimension represents deterioration. After the input information is imported into the prediction model, the two-dimensional vector is obtained. More preferably, normalization is performed on the two-dimensional vector by using the softmax function, so that the two-dimensional vector is converted into a probability of non-deterioration (a first dimension value) and a probability of deterioration (a second dimension value), and the probability of non-deterioration and the probability of deterioration are added to equal 1. Optionally, it may be determined, according to a dimension with a relatively large value, that a corresponding sample is classified into a corresponding prediction result (deterioration or non-deterioration).

Based on the above-mentioned description, according to the method for establishing a prediction model of the present invention, a standard model set/meta-classifier including single-modality standard models and multi-modalities standard models is established in step S1 and step S2, and corresponding to inputted training data, standard models are correspondingly imported from the standard model set in step S3, to train the prediction model of the present invention. Therefore, in the present invention, the standard model set is established by considering various modality combinations, compared with the related art in which only a single standard model is used (without considering standard models including a plurality of modality combinations), the prediction model can be more completely and more accurately trained. Therefore, in the present invention, the established prediction model can generate a reliable prediction result for different input data (input data including at least one type of modalities to complete types of modalities, that is, any one of single-modality data, multi-modalities data and complete-modalities data). Conversely, in the related art, a prediction result can be generated under only complete types of modalities or an inaccurate prediction result is generated under incomplete types of modalities.

Particularly, when the multi-modalities standard models are established in step S2, Cₘ^{N} modality combinations having m types of the modalities are obtained from the samples having a plurality of modalities, data of complete types of modalities or data of a relatively large quantity of types of modalities may be used to form modality data having a target combination. In addition, a corresponding multi-modalities standard model may be established according to the modality data having only the target combination. For example, in samples having four types of modalities at most, if a standard model needs to be established for only a first type modality M1 and a second type modality M2, non-related types of modalities may be removed from samples having complete types of modalities [M1, M2, M3, M4] (that is, a third type modality M3 and a fourth type modality M4 are replaced by 0), to form a corresponding data type [M1, M2, 0, 0], or non-related types of modalities may be removed from samples having a relatively large quantity of types of modalities [M1, M2, M3, 0] and [M1, M2, 0, M4] to form a corresponding data type [M1, M2, 0, 0], and samples with an original data type of [M1, M2, 0, 0] can be also applied to establish a multi-modalities standard model for a modality combination [M1, M2, 0, 0]. Therefore, more sample data may be generated to establish standard models, to improve credibility of the corresponding standard models, thereby improving credibility of a subsequent prediction model.

Preferably, after the prediction model is established, correctness of a corresponding prediction result of a corresponding modality in a corresponding sample may be continuously tracked and verified. For example, a verification result is generated by using real statistical data, or a verification result is generated by performing evaluation by an expert in a corresponding field, and the verification result is fed back to a corresponding prediction system to correct/modify the corresponding prediction model. The verification result may correspond to a factor or a parameter of each modality in the prediction model, for example, a weight of each modality for the prediction result may be calculated by using various calculation methods or calculation models to adjust/modify the prediction model.

The pre-established standard model, especially, the single-modality standard model, is configured to generate a corresponding prediction result (a modality) after corresponding characterization information (clinical factor, brain image, electroencephalography, environment air pollution, gene, or the like) is inputted and is applicable to a prognostic system for Alzheimer's disease, to assist in determining whether the clinical outcome of a patient with Alzheimer's disease is improving or worsening. In other words, the prognostic system for Alzheimer's disease includes the prediction system/prediction model, where multiple types of modalities of interest corresponding to input information are selected from any three types of a clinical factor modality, a brain image modality, an electroencephalography modality, an environment air pollution modality and a gene modality. Each of the multiple types of modalities of interest is generated by inputting corresponding characterization information into a corresponding single-modality standard model. The corresponding characterization information correspondingly refers to at least three of the clinical factor characterization information, the brain image characterization information, the electroencephalography characterization information, the environment air pollution characterization information and the gene characterization information. The corresponding single-modality standard model correspondingly refers to at least three of a clinical factor standard model, a brain image standard model, an electroencephalography standard model, an environment pollution standard model and a gene standard model. Therefore, by using the prognostic system for Alzheimer's disease established by using the prediction model, in the absence of input data/modalities (there are only partial/incomplete input data/modalities), a stable and credible prediction result can be still obtained. As the above-mentioned, the state in the absence of input data/modalities means that there are only partial/incomplete input data/modalities; particularly indicating the state that the complete modalities have N types of modalities, and the input data has a modality/modalities with types less than N.

For the clinical factor standard model, a corresponding prediction result is generated according to corresponding clinical factor characterization information and used as the clinical factor modality. The clinical factor characterization information is one or more of demographic variables (for example, an information of age and gender), a history of related diseases and comorbidities, brain cognitive functions and mental behavior symptoms of a patient. In the history of related diseases and comorbidities, the presence or absence of specific risk factors (for example, diabetes, hypertension, stroke, and hyperlipidemia, but are not limited to the above diseases) need to be considered, and the variables (for the presence of specific risk factors) are considered and calculated. At least two cognitive functions are selected from the brain cognitive functions for calculation. The cognitive functions include short-term memory, long-term memory, attention, orientation, drawing, abstract thinking and judgement, verbal fluency, and language, and continuous variable scores corresponding to the cognitive functions are defined. At least one mental behavior is selected from the mental behavior symptoms for calculation. The mental behaviors include: one of more of behaviors such as hallucinations, delusions, agitation/aggression, depression, anxiety, irritability, disinhibition, euphoria, apathy, aberrant motor behavior, sleep and night-time behavior change, and appetite and eating changing. Each behavior may be represented by factors such as a frequency of occurrence, severity, and a degree of trouble (for example, represented by a value) in a form of a continuous variable. By using the above-mentioned considered factors, a corresponding preset standard model is established based on a machine learning method.

For the brain image standard model, a corresponding prediction result is generated according to corresponding brain image characterization information and used as the brain image modality. The brain image characterization information is magnetic resonance image (MRI) or a computed tomography (CT) image of a brain of a patient. In the MRI, image registration is performed on a brain image of the patient and a brain template by using a 3D high resolution T1-weighted image such as an image with an image voxel of 1×1×1 mm³, and then image segmentation is performed to divide the brain image into three parts including a gray matter, a white matter, and a cerebrospinal fluid (CSF). After different gray matter regions with a quantity of Ng and different white matter regions with a quantity of Nw are defined, volumes of these regions are calculated, and shape features with a quantity of Ns and texture features with a quantity of Nt in the regions are analyzed by using, for example, free software PyRadiomics to obtain a plurality of analysis parameters (for example, shape features, first order parameters, grey-level co-occurrence matrix (GLCM) features, grey-level dependency matrix (GLDM) features, and neighborhood gray-tone difference matrix (NGTDM) features). Ng, Nw, Ns, and Nt are all positive integers. Therefore, gray matter volume values of the Ng regions, white matter volume values of the Nw regions, gray matter shape features with a quantity of a product calculated by Ng and Ns (Ng×Ns), gray matter texture features with a quantity of a product calculated by Ng and Nt (Ng×Nt), white matter shape features with a quantity of a product calculated by Nw and Ns (Nw×Ns), and white matter texture features with a quantity of a product calculated by Nw and Nt (Nw×Nt) may be obtained in total.

In the CT image, a brain range is divided by using a Unet network, and an intracranial brain volume, a brain parenchyma volume, and a brain parenchyma volume proportion are calculated. The brain parenchyma volume proportion is equal to the brain parenchyma volume divided by the intracranial brain volume and the brain parenchyma volume proportion is used as a parameter of brain atrophy. Subsequently, the brain template image is aligned to the CT image, and brain gray and white matter regions are directly segmented by using a brain standard template. For example, a brain region may be divided into Ng brain gray matter regions and Nw brain white matter regions by using a Johns Hopkins University (JHU) brain region template used in public or an anatomical automatic labeling (AAL) template and through image spatial transformation, and then shape and texture analysis are performed on each region by using the free software PyRadiomics, to obtain Ns shape features and Nt texture features, so that parameters of Ng region gray matter volume values, Nw white matter volume values, Ng×Ns gray matter shape features, Ng x Nt gray matter texture features, Nw×Ns white matter shape features, and Nw×Nt white matter texture features can be obtained in total.

Based on the feature parameters obtained from said MRI and said CT image, and basic physiological parameters such as age and gender of the patient are optionally added/considered, a corresponding preset standard model is established by using the machine learning method or architecture such as a support vector machine (SVM), a decision tree, a least absolute shrinkage and selection operator (LASSO), or ensemble learning.

For the electroencephalography standard model, a corresponding prediction result is generated by the electroencephalography characterization information, including at least one feature of electroencephalography with a specific frequency and localization, and used as the electroencephalography modality. For the frequency feature of the electroencephalography characterization information may be one of an alpha wave within frequency 8-13 Hz, a theta wave within frequency 4-7 Hz, a beta wave within frequency 14-20 Hz and a delta wave within frequency 1-3 Hz. For the localization, particularly corresponding to said types of waves within the specific frequencies, of the electroencephalography characterization information may be at least one of a frontal area, a temporal area, an occipital area and a parietal area. A corresponding preset standard model is established based on the machine learning method.

For the environment air pollution standard model, a corresponding prediction result is generated according to corresponding environment air pollution characterization information and used as the environment air pollution modality. The environment air pollution characterization information is air pollution data of a life place (for example, a residential place, a working place, a living place, and other positions) of the patient. The air pollution data includes at least one of concentration of suspended particulates (PM10 or lower), concentration of fine suspended particulates (PM2.5 or lower), concentration of nitrogen oxides, concentration of nitrogen monoxide, concentration of nitrogen dioxide, concentration of carbon monoxide, concentration of carbon dioxide, or concentration of ozone. A risk prediction model of the air pollution data for occurrence and progression of Alzheimer's disease is analyzed by using a method such as case-control study or cohort study, and estimation is performed by using a time sequence econometrics statistical method, and a corresponding preset standard model is established based on the machine learning method.

For the gene standard model, a corresponding prediction result is generated according to corresponding gene characterization information to form the gene modality. The gene characterization information is a single nucleotide polymorphism (SNP) at a specific gene site, for example, an apolipoprotein E (APOE) gene. In the APOE gene, classification may be performed by considering one of E2/E2, E2/E3, E2/E4, E3/E3, E3/E4, and E4/E4 or according to absence or presence of E4 allele. Optionally or alternatively, the gene characterization information may also be a length change of nucleotides. For example, any one of an II gene type, an ID gene type, or a DD gene type of ACE gene is used as a representative, and the corresponding gene type is converted into a corresponding value and weight. Weights of the SNP at the gene site and the length data of the nucleotide may be calculated and converted into corresponding values. The corresponding preset standard models are established by using the above-mentioned considered factors, value conversion, and weight application and based on the machine learning method.

Based on the single-modality standard models established by using the at least three of the clinical factor characterization information, the brain image characterization information, the electroencephalography characterization information, the environment air pollution characterization information and the gene characterization information, and correspondingly based on the clinical factor modality, the brain image modality, the electroencephalography modality, the environment air pollution modality or the gene modality generated by the single-modality standard models, the corresponding multi-modalities standard models and the prediction model for prognosis of Alzheimer's disease may be established by using step S1 to step S3 of the method for establishing a prediction model of the present invention, and the corresponding prediction system for prognosis of Alzheimer's disease is established by using the obtained prediction model.

In summary, according to the method for establishing a prediction model of the present invention, a standard model set including single-modality standard models and multi-modalities standard models can be established, and corresponding standard models are imported according to different input data, to train a prediction model, so that the prediction model can be more completely and more accurately trained. Therefore, a reliable prediction result can be generated for the input data of different modality combinations. In addition, when the multi-modalities standard models are established, a plurality (Cₘ^{N}) of modality combinations having a plurality (m) of modalities are obtained from the sample having a plurality of modalities. Wherein, data of complete modalities, or data of a relatively large quantity of modalities, or data of exact modalities/modality may be used to form modality data having a target combination to establish a corresponding multi-modalities standard model, so that more samples/modality data may be generated for establishing standard models, to improve credibility of the corresponding standard models, thereby improving credibility of a subsequent prediction model. In addition, through the prediction system established by using the prediction model, in the situation that input data/modalities are incomplete, a stable and credible prediction result can be still obtained. In addition, through the prediction system for Alzheimer's disease established by using the prediction model, in the situation that input data/modalities are incomplete, a stable and credible prediction result can also be obtained.

Although the present invention has been disclosed above through the preferred embodiments, the embodiments are not intended to limit the present invention. Various changes and modifications made by any person skilled in the art to the embodiments without departing from the spirit and scope of the present invention are still within the protective technical scope of the present invention. Therefore, the protection scope of the present invention should include meanings recorded in the attached claims and all changes within the equivalent scope. In addition, when the plurality of embodiments can be combined, the present invention includes any combination of embodiments.

What is claimed is:

1. A method for establishing a prediction model, performed by a computer, wherein the computer comprises at least one processor and at least one storage unit coupled to the processor, the storage unit comprises multiple samples, each of the samples comprises at least one type of modalities, the samples have N types of modalities in total, and some of the samples each has N types of modalities simultaneously, wherein N is a positive integer not less than 3; and the processor performs the following steps:
respectively obtaining C₁^{N} pre-established single-modality standard models according to every single type of the modalities in the samples;
obtaining Cₘ^{N} modality combinations each having m types of the modalities from the samples having multiple types of the modalities to establish Σₙ₌₂^{N-1}Cₙ^{N} corresponding multi-modalities standard models, wherein m is a combination of positive integers not greater than N−1 and not less than 2;
obtaining Σₙ₌₁^{N}Cₙ^{N} modality combinations from the samples having N types of modalities simultaneously to be training data, wherein the modality combinations in the training data can be classified as single-modality training data, multi-modalities training data, and complete-modalities training data; the single-modality training data has C₁^{N} modality combinations in total and the modality combinations have different single types of modalities from each other; the multi-modalities training data has greater than or equal to 2 types and less than or equal to N−1 types of the modalities and has Σₙ₌₂^{N-1}Cₙ^{N} modality combinations in total, the modality combinations have different multiple types of modalities from each other; the complete-modalities training data has a C_{N}^{N} modality combination and has N types of modalities simultaneously; inputting the training data into a to-be-trained prediction model, and modifying the to-be-trained prediction model by using the single-modality standard models and the multi-modalities standard models, to obtain a trained prediction model.

2. The method for establishing a prediction model according to claim 1, wherein each of the samples which are used to be the training data has a corresponding ground truth; when the inputted training data is the single-modality training data or the multi-modalities training data, a ground truth of a corresponding one of the samples is imported, each of the single types of the modalities or each of the multiple types of the modalities is calculated by using the to-be-trained prediction model to output a prediction result, a corresponding training data is inputted into a corresponding one of the single-modality standard models or a corresponding one of the multi-modalities standard models to generate a corresponding standard result, and a corresponding loss function is calculated by using the prediction result, the ground truth and the standard result; and when the inputted training data is the complete-modalities training data, each modality in the complete-modalities training data is calculated by the to-be-trained prediction model to output a prediction result, a ground truth of a corresponding one of the samples is imported, and a corresponding loss function is calculated by using the prediction result and the ground truth.

3. The method for establishing a prediction model according to claim 2, wherein when the inputted training data is the single-modality training data or the multi-modalities training data, the loss function is defined by a classification loss and a distillation loss; the corresponding classification loss is calculated by using a corresponding prediction result and a corresponding a ground truth; and the corresponding distillation loss is calculated by using a corresponding prediction result and a corresponding standard result.

4. The method for establishing a prediction model according to claim 2, wherein when the inputted training data is the complete-modalities training data, the loss function is defined by a classification loss; and the corresponding classification loss is calculated by using a corresponding prediction result and a corresponding ground truth.

5. The method for establishing a prediction model according to claim 2, wherein the loss function can be defined as follows:
$${arg min}_{θ} {\sum }_{i}^{c} [ {\sum }_{j}^{{\{ {C}_{hn} \}}_{hn = 1 ∼ N}} {l}_{c l {s}_{j}} ( {\{ {X}_{i}^{k C} \}}_{k = j} , {y}_{i} ; θ ) +$$
$${\sum }_{s}^{{\{ {C}_{h m} \}}_{h m = 1 ∼ m}} {α}_{s} {l}_{{d}_{s}} ( {\{ {X}_{i}^{tC} \}}_{t = s} ; T {e}_{s} ( {ω}_{s} ) ) ]$$
$$wherein {argmin}_{θ} ( θ )$$
is used for representing that a parameter value 9 of the prediction model is calculated by reducing the current loss function to a minimum value; c is used for representing a quantity of samples having complete modalities in training data; i is used for representing a training data sample index value being considered in an epoch; yᵢ is used for representing a ground truth in the training data with a corresponding sample index value; N is used for representing a total quantity of types of modalities; Cₕₙ is used for representing combinations of hn types of modalities selected from N modalities; {Cₕₙ}_{hn=1˜N} is used for representing a set of the combinations of the hn types of modalities from the N types of modalities; j is used for representing a modality combination index value being considered in a classification loss function; l_{cls}_{ⱼ} is used for representing a classification loss function used for calculating a cross entropy loss between an output of a prediction model and ground truth yᵢ, and a subscript j thereof represents a considered modality index value; {Xᵢ^{kC}}ₖ₌ⱼ is used for representing a modality combination j in an i^{th} sample having complete modalities in the training data; θ is used for representing a model parameter of the prediction model; m is used for representing a maximum quantity of the multiple types of the modalities; Cₕₘ is used for representing combinations of hm types modalities selected from m types of modalities; {Cₕₘ}_{hm=1˜m} is used for representing a set of the combinations of the hm types of modalities selected from the m types of modalities; s is used for representing a modality combination index value being considered in a distillation loss function; l_{d}_{ₛ} is used for representing a distillation loss function, wherein a subscript s thereof represents a considered modality index value; {Xᵢ^{tC}}ₜ₌ₛ is used for representing a modality combination s in the i^{th} sample having the complete modalities in the training data; Teₛ(ωₛ) is used for representing a standard model adapted for calculating the modality combination s; ωₛ is used for representing a model parameter of the standard model adapted for calculating the modality combination s; and αₛ is used for representing a ratio value of each standard model of a corresponding one of modality combinations s in the distillation loss function to an overall loss function of the prediction model, wherein the value is a positive number not less than 0

6. The method for establishing a prediction model according claim 2, wherein based on a teacher-student model training architecture, the single-modality standard models and the multi-modalities standard models are teacher models, and the prediction model is a student model.

7. The method for establishing a prediction model according to claim 1, wherein in a process of establishing the multi-modalities standard models, the multi-modalities standard models are trained by using a loss function and a gradient descent method, a parameter of a corresponding one of the multi-modalities standard models is modified in each epoch to minimize the loss function, and the trained multi-modalities standard models are established after specified epochs are completed.

8. A prediction system, comprising:
at least one processor; and
at least one storage unit coupled to the processor, wherein the storage unit has a prediction model, the prediction model is established by using the method for establishing a prediction model according to any one of claims 1 to 7 so as to generate a prediction result for one or more of multiple types of modalities of interest in an input information; and the processor receives the input information having the one or more of the multiple types of modalities of interest, and the processor imports the input information to the prediction model to generate the corresponding prediction result.

9. A prognostic system for Alzheimer's disease, comprising the prediction system according to claim 8, wherein the multiple types of modalities of interest in the input information are selected from any three types of a clinical factor modality, a brain image modality, an electroencephalography modality, an environment air pollution modality and a gene modality; each of the multiple types of modalities of interest is generated by inputting a corresponding characterization information into a corresponding one of the single-modality standard models; the corresponding characterization information refers to at least three of a clinical factor characterization information, a brain image characterization information, an electroencephalography characterization information, an environment air pollution characterization information and a gene characterization information; and the corresponding single-modality standard model refers to at least three of a clinical factor standard model, a brain image standard model, an electroencephalography standard model, an environment pollution standard model and a gene standard model.

10. The prognostic system for Alzheimer's disease according to claim 9, wherein the clinical factor characterization information comprises at least one of an information of age and gender, a history of related diseases and comorbidity, brain cognitive functions and mental behavior symptoms of a patient; the brain image characterization information comprises a magnetic resonance image or a computed tomography image of a brain of a patient; the electroencephalography characterization information includes at least one feature of electroencephalography with a specific frequency and localization; the environment air pollution characterization information is air pollution data of a life place of a patient, wherein the air pollution data comprises at least one of concentration of suspended particulates, concentration of fine suspended particulates, concentration of nitrogen oxide, concentration of nitrogen monoxide, concentration of nitrogen dioxide, concentration of carbon monoxide, concentration of carbon dioxide and concentration of ozone; and the gene characterization information is single nucleotide polymorphism at a specific gene site and/or a length of nucleotide.
